(19) United States
(12) Patent Application Publication
(10) Pub. No.: US 2025/0308025 A1
(43) Pub. Date: Oct. 2, 2025

(54) REFERENCE IMAGE PROVIDING APPARATUS, REFERENCE IMAGE PROVIDING METHOD, REFERENCE IMAGE PROVIDING PROGRAM, AND REFERENCE IMAGE PROVIDING SYSTEM

(71) Applicant: KONICA MINOLTA, INC., Tokyo (JP)

(72) Inventors: Yuki TOMIYAMA, Tokyo (JP); Kazuhiro ONO, Osaka (JP); Masaki SUNAGAWA, Osaka (JP)

(73) Assignee: KONICA MINOLTA, INC., Tokyo (JP)

(21) Appl. No.: 19/089,372

(22) Filed: Mar. 25, 2025

(30) Foreign Application Priority Data
Mar. 26, 2024 (JP) 2024-049524

(51) Int. Cl. G06T 7/00; G16H 50/20

(52) U.S. Cl. CPC G06T 7/0014; G16H 50/20; G06T 2207/30004; G06T 2207/30196

(57) ABSTRACT

A reference image providing apparatus capable of reducing user's labor required for registration of a reference image, wherein the reference image providing apparatus includes an extractor configured to extract a reference image or a candidate image of the reference image from diagnostic images used for diagnosis, the reference image serving as an imaging reference for determining whether or not an image can be used for diagnosis; and a provider configured to provide an extraction result. For example, when the reference image is not set, the extractor extracts the reference image or the candidate image.

### CROSS-REFERENCE TO RELATED APPLICATION

This application claims the benefit of priority to Japanese Patent Application No. 2024-049524 filed on 26 Mar. 2024, the entire disclosure of which is incorporated herein by reference in its entirety.

### TECHNICAL FIELD

The present invention relates to a reference image providing apparatus, a reference image providing method, a reference image providing program, and a reference image providing system.

### DESCRIPTION OF RELATED ART

In the medical field, various image inspections are performed on a patient using an inspection apparatus such as a radiation imaging apparatus.

When capturing a medical image, there may be a case in which the medical image is determined to be unsuitable for diagnosis due to a defect of an imaged part, a body motion, or the like, and in such a case a re-capturing is performed.

Conventionally, there exists a system, as a rejected image management system, which is capable of displaying a rejected image, a diagnostic image, and a reference image side by side (see, for example, Japanese unexamined patent publication No. 2022-155771).

The rejected image is a no good (NG) image that has failed to be captured and is not used for diagnosis.

The diagnostic image is a good (OK) image that is successfully captured and is used for diagnosis.

The reference image is an image that is suitable for diagnosis and is a goal for imaging.

Using this system, for example, it is possible to train a radiographer about an X-ray imaging technique while making the radiographer view the rejected image, the diagnostic image, and the reference image displayed side by side. The reference image is registered in advance for each imaging method by a person (for example, an imaging technician or a doctor in each medical facility).

### SUMMARY OF INVENTION

### Technical Problem

However, the conventional system requires a person to select a reference image in advance from a large number of images for each imaging method, which needs a lot of time and effort, and thus is not surely convenient.

The present invention is made in view of the above-described problem, and an object of the present invention is to provide a reference image providing apparatus, a reference image providing method, a reference image providing program, and a reference image providing system that can reduce user's labor required for registration of the reference image.

### Solution to Problem

To achieve the abovementioned object, according to an aspect of the present invention, one aspect of the present invention includes a reference image providing apparatus including: an extractor configured to extract a reference image or a candidate image of the reference image from diagnostic images used for diagnosis, the reference image serving as an imaging reference for determining whether or not an image can be used for diagnosis; and a provider configured to provide an extraction result.

### BRIEF DESCRIPTION OF DRAWINGS

The advantages and features provided by one or more embodiments of the invention can be fully understood from the detailed description given hereinbelow and the appended drawings which are given by way of illustration only and thus are not intended to define the limits of the present invention, in which:

FIG. 1 is a configuration diagram of a rejected image management system according to an embodiment of the present invention;

FIG. 2 is a diagram showing an example of data structure of imaging result record information;

FIG. 3 is a block diagram illustrating a configuration of a rejected image management apparatus according to the embodiment of the present invention;

FIG. 4 is a diagram illustrating an example of data configuration of an image management table;

FIG. 5 is a diagram illustrating an example of data configuration of a reference image setting table;

FIG. 6 is a block diagram illustrating functions related to rejected image management implemented by the rejected image management apparatus according to the embodiment of the present invention;

FIG. 7 is a diagram showing an example of a reference image setting screen;

FIG. 8 is a diagram showing an example of a comparison display screen;

FIG. 9 is a diagram showing an example of processing when the rejected image management apparatus according to the embodiment of the present invention acquires a diagnostic image; and

FIG. 10 is a diagram illustrating an example of processing when the rejected image management apparatus according to the embodiment of the present invention is instructed to display the comparison display screen 7.

### DETAILED DESCRIPTION OF THE EMBODIMENTS

Hereinafter, embodiments of the present invention are described in detail with reference to the drawings. However, the scope of the invention is not limited to the disclosed embodiments.

Each of the drawings is merely schematically illustrated so that the present invention can be fully understood.

Accordingly, the present invention is not limited to only the examples illustrated in the drawings. Further, in the respective drawings, common components and similar components are denoted by the same reference signs, and duplicated description thereof is omitted. Furthermore, detailed description of well-known functions that are not related directly to the present invention may be omitted.

### Configuration of Rejected Image Management System According to Embodiment

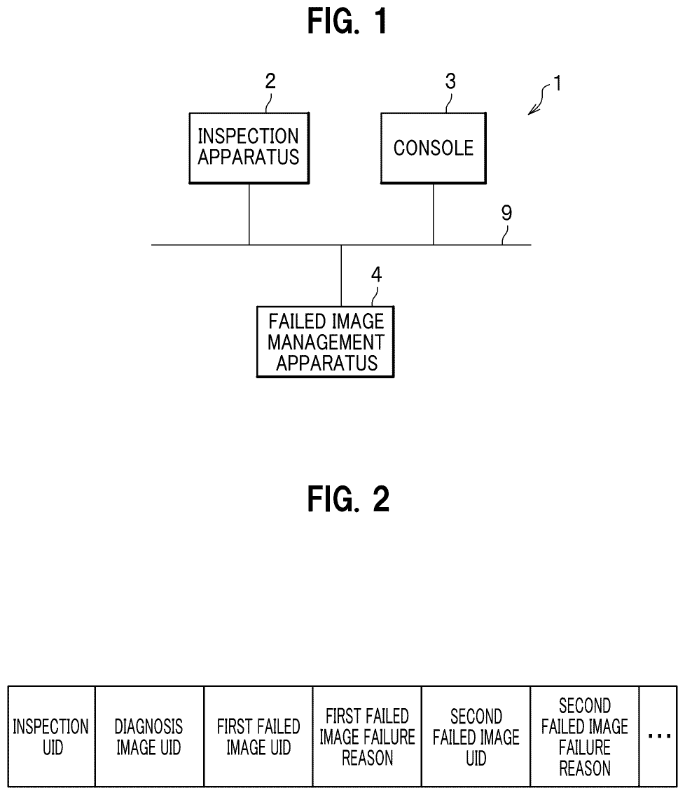

FIG. 1 is a configuration diagram of a rejected image management system 1 according to the embodiment.

A rejected image management system 1 illustrated in FIG. 1 is a system managing a rejected image that occurs in capturing a medical image.

The rejected image management system 1 may be included in a medical image management system (PACS: Picture Archiving and Communication System).

In addition, the rejected image management system 1 may be able to transmit data to and receive it from another system (for example, a reporting system).

The rejected image is an NG image due to its failing to be properly imaged for some reason.

In a case in which several images have been determined to be rejected and then imaging is performed many times for scheduled imaging of one image (for the same examination of the same patient), which results in a plurality of rejected images.

The diagnostic image is an OK image that is successfully captured and is used for diagnosis.

The diagnostic image may be an image (re-captured image) that has been re-captured due to imaging failure.

The reference image is an image suitable for diagnosis and is an image serving as an imaging reference. The reference image is set in advance for each imaging method.

Each medical facility can define its reference image as an imaging reference required in the medical facility.

Note that the reference image is such as a goal defined by a medical facility, but is not an image indicating a boundary criterion for determining whether an image falls into a diagnostic image or a rejected image.

That is, each medical facility determines whether or not a medical image is usable for diagnosis, and therefore, even if the medical image is not as good for a reference image, it can be determined to be a diagnostic image.

The reference image is often specified for each imaged part and imaging direction, but can also be specified by further adding factors affecting imaging, such as a physique of a patient.

Further, a plurality of reference images can be designated for the same imaging method.

For example, the radiographer performs imaging with reference to the reference image in the case of a patient who is imaged for the first time.

Furthermore, the reference image is used by a doctor who gives a lecture to a radiographer about how to capture an image in an educational conference or the like.

As shown in FIG. 1, the rejected image management system 1 includes an inspection apparatus 2, a console 3, and a failed image management apparatus 4.

The respective apparatuses included in the rejected image management system 1 are connected to each other so as to be able to transmit and receive data via a communication network 9 such as a local area network (LAN).

The apparatuses included in the rejected image management system 1 are compliant with “Health Level Seven (HL7)” or “Digital Imaging and Communication in Medicine (DICOM) standard”, and communication between the apparatuses is performed in accordance with “HL7” or “DICOM”.

The inspection apparatus 2 is a modality such as an X-ray imaging apparatus (DR or CR), an ultrasound diagnostic apparatus (US), a computed tomography (CT), or a Magnetic Resonance Imaging (MRI), and captures an image of a patient to generate a medical image.

The medical image generated by the inspection apparatus 2 is image data expressed in digital.

The inspection apparatus 2 attaches supplementary information to a medical image by writing the supplementary information in a header of an image file of the medical image in accordance with the DICOM standard.

The supplementary information includes, for example, a patient ID, name, sex, date of birth, inspection UID (Unique Identifier), imaging region, imaging direction, imaging date and time, imaging technician, modality, tube voltage, tube current, irradiation time, and image UID.

The patient ID is identification information for identifying a patient.

The inspection UID is identification information for identifying an inspection.

The image UID is identification information for identifying a medical image.

The console 3 controls an imaging operation in the inspection apparatus 2 in accordance with an imaging order (including patient information such as a patient ID and a patient name and imaging request information such as an imaging region and an imaging direction).

The console 3 includes a display part that displays a medical image captured by the inspection apparatus 2 and an operation part that receives an operation input from an imaging technician.

The imaging technician performs various kinds of setting relating to inspection through input from the operation part of the console 3.

The imaging technician uses the console 3 to check whether the medical image captured by the inspection apparatus 2 falls into a diagnostic image or a rejected image.

Note that the console 3 may automatically determine whether the medical image falls into a diagnostic image or a rejected image based on image analysis or the like.

In a case in which the imaging technician determines that the medical image obtained by imaging falls into the rejected image, the imaging technician records that the medical image is the rejected image and a reason for the rejection through operation at the operation part of the console 3.

There are many kinds of reasons why an imaging failure occurs, and examples of the reasons include positioning, body motion, a foreign substance, insufficient inhalation, insufficient dose, an artifact, catheter readjustment, imaging conditions, mistaken patient, mistaken body part, and the like.

If the imaging technician determines that the medical image obtained by imaging is a failed image, the imaging technician re-captures a medical image.

The re-captured image is an image obtained by re-capturing the same patient with respect to the same imaging order as those of the failed image.

The re-captured image is obtained by the same imaging method as that of the failed image.

The imaging method includes an imaging part, an imaging direction, an imaging technique, and the like, or may include conditions related to an imaging target.

On the other hand, among imaging conditions, the tube voltage, the tube current, the irradiation time, and the like are adjusted at the time of re-capturing, so that values of such conditions may be different between the failed image and the re-captured image.

The console 3 generates imaging result record information for each inspection through an operation by an imaging technician based on an imaging order.

The imaging result record information is information indicating a correspondence between the diagnostic image and the failed image for an inspection identified by the inspection UID.

The imaging result recording information includes at least the inspection UID and the image UID of the diagnostic image.

The console 3 transmits to the failed image management apparatus 4 the imaging result record information generated for each inspection together with the image file of the medical image.

FIG. 2 is an example of data structure of the imaging result record information.

The inspection UID is identification information for identifying an inspection.

The inspection UID specifies a patient to be inspected and content of an inspection (the present embodiment assumes imaging for the content).

In the present embodiment, an inspection to be performed according to a certain imaging order is assumed to be managed by a single inspection UID.

That is, when a certain inspection is performed on a certain patient, both a failed image whose imaging has failed and a diagnostic image to be used for diagnosis are managed with the same inspection UID.

The image UID is identification information for specifying a medical image, and for example, a Service Object Pair (SOP) instance UID or the like is used.

The image UID of the diagnostic image is an image UID of an image (diagnostic image) that has been successfully captured in the inspection indicated by the inspection UID.

When the diagnostic image is a re-captured image, the imaging result record information further includes image UID of the k-th failed image and imaging failure reason of the k-th failed image by the number of times of reimaging (k=1, 2, . . . , n (n is a natural number)).

The failed image management apparatus 4 illustrated in FIG. 1 is a computer that manages the medical images captured by the inspection apparatus 2, information on failed images (information such as information indicating whether or not each medical image is a failed image, and a correspondence between a diagnostic image and a failed image), and the like.

The failed image management apparatus 4 according to the present embodiment has a function of extracting and providing a reference image serving as an imaging reference for determining whether or not an image can be used for diagnosis from a captured medical image, or a candidate image of the reference image can be extracted and provided in place of the reference image.

That is, the failed image management apparatus 4 works as a “reference image providing apparatus” that automatically extracts and provides a reference image (or a candidate image) without depending on a human operation.

The failed image management apparatus 4 is an example of a “reference image providing apparatus”.

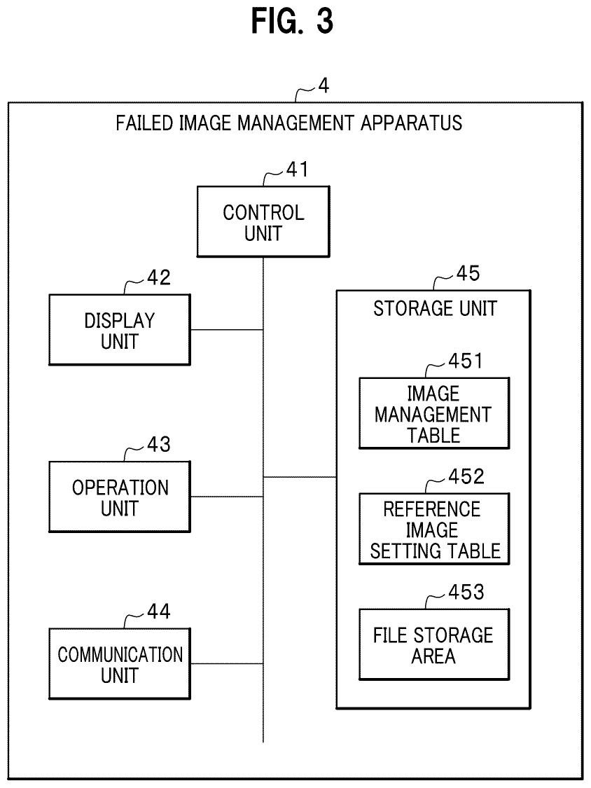

An arrangement of the failed image management apparatus 4 is described with reference to FIG. 3 (see FIG. 1 as needed).

As shown in FIG. 3, the failed image management apparatus 4 includes a control unit 41, a display unit 42, an operation unit 43, a communication unit 44, a storage unit 45, and the like, which units are connected to each other via a bus.

The control unit 41 includes a Central Processing Unit (CPU), a Random Access Memory (RAM), and the like, and integrally controls a processing of each unit of the failed image management apparatus 4.

Specifically, the CPU reads various processing programs stored in the storage unit 45, develops the programs in the RAM, and performs various kinds of processing with cooperation with the programs.

The display unit 42 includes a monitor such as a liquid crystal display (LCD), and displays various screens according to an instruction of a display signal input by the control unit 41.

The operation unit 43 includes a keyboard including cursor keys, character/number input keys, and various function keys, and a pointing device such as a mouse, and outputs to the control unit 41 an operation signal input through a key operation on the keyboard or a mouse operation.

The communication unit 44 includes a network interface and the like to transmit to and receive from data an external apparatus connected via the communication network 9 (see FIG. 1).

For example, the communication unit 44 receives, from the console 3 (see FIG. 1), a medical image obtained by imaging a patient using the inspection apparatus 2 and imaging result record information.

The storage unit 45 includes a nonvolatile memory or the like (e.g., a hard disk drive (HDD) or a solid-state drive (SSD)) and stores various kinds of data required for managing failed images.

The storage unit 45 stores an image management table 451 and a reference image setting table 452.

The storage unit 45 has a file storage area 453.

The image management table 451 stores information on each medical image stored in the file storage area 453.

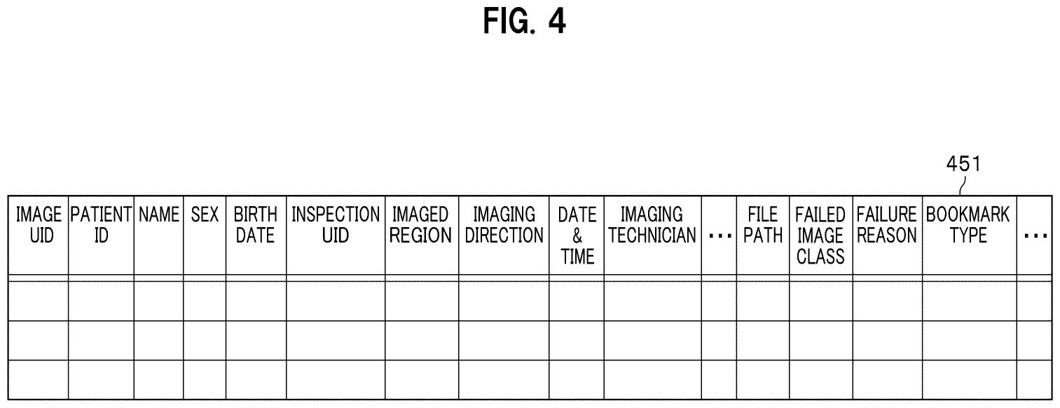

FIG. 4 is a diagram illustrating an example of a data configuration of an image management table 451.

The image management table 451 associates, for each medical image, an image UID, a patient ID, a name, sex, a date of birth, an inspection UID, an imaging part, an imaging direction, an imaging date and time, an imaging technician, a file path, a failed image classification, a failed image reason, a bookmark type, and the like.

The image UID of the medical image is stored in the “image UID” field.

The “patient ID”, “name”, “sex”, and “date of birth” fields store, respectively, a patient ID, name, sex, and date of birth of a patient to be imaged.

The “inspection UID” field stores an inspection UID of an inspection related to a medical image.

The “imaging region”, “imaging direction”, “imaging date and time”, and “imaging technician” fields stores respectively an imaging region, an imaging direction, an imaging date and time, and an imaging technician of the medical image.

Information such as the image UID, the patient ID, the name, the sex, the date of birth, the inspection UID, the imaging part, the imaging direction, the imaging date and time, and the imaging technician is acquired from supplementary information of the medical image.

The “file path” field stores a storage location of the medical image in the file storage area 453.

The “failed image classification” field stores information indicating whether the medical image is a diagnostic image or a failed image (failed image classification).

When the medical image is a failed image, the failed image classification includes information indicating how many times the failed image is re-captured.

For example, the failed image classification is set to “0” in a case of the diagnostic image, “1” in a case of the first failed image, and “2” in a case of the second failed image, . . . and so on.

The “failed image reason” field stores an imaging failure reason when the failed image classification is other than “0”, that is, when the medical image is a failed image.

When a medical image is classified by a bookmark function, a bookmark type is stored in the “bookmark type” field.

The bookmark function is a function of adding a bookmark to an image of interest (an image of high interest) during image checking.

Tagging a specific medical image makes it possible to specify a bookmark type and search for and extract a medical image belonging to the specified bookmark type when the medical image is viewed again later.

The bookmark type includes, for example, for a conference (an image to be used in a next conference), for a conference presentation, and for an important inspection.

A name of the bookmark type can be defined in advance by a user, and the following bookmark type can also be used: “a most difficult knee image that has failed to be captured”; “an image on which a user wants to ask an experienced person to check”; “an image which has greatly improved an imaging technique”, or the like.

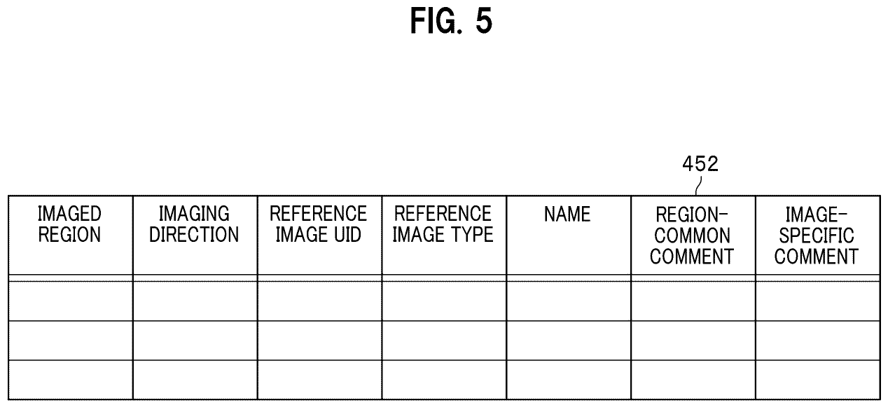

The reference image setting table 452 illustrated in FIG. 3 is a table for managing reference images, which table is configured for respective imaging methods (including an imaging part and an imaging direction). FIG. 5 is a diagram illustrating an example of a data configuration of the reference image setting table 452.

The reference image setting table 452 associates an image UID of a reference image, a reference image type, a name, a region-common comment, and an image-specific comment with a combination of an imaging part and an imaging direction.

The “imaged region” and an “imaging direction” are an imaging method in which the reference image is designated.

The “image UID of reference image” is an image UID of a medical image specified as the reference image.

The “reference image type” is a type of the reference image and includes the “A reference” and the “B reference”.

The A reference is an image which is very suitable as a reference image (for example, the best image in the same photographing method).

The B reference is an image suitable as a reference image (e.g., an image at a passing level or an acceptable level).

The setting criterion of the reference image is different for each medical facility.

Although the reference images are classified into two types here, the reference images may be classified into more types than two and the reference image type may be set with one of the more types, and there may be only one type of reference image.

The “name” is a name of the reference image.

The “region common comment” is a comment input for the imaging region of the reference image.

The “image specific comment” is a comment input for the reference image.

The region-common comment and the image-specific comment are input as a memo or a message when the reference image is set or referred to.

Note that a reference image may be allowed to be searched for using a term included in the comment.

The control unit 41 illustrated in FIG. 3 implements various functions related to failed image management (including a function of providing a reference image) by executing a program using the CPU.

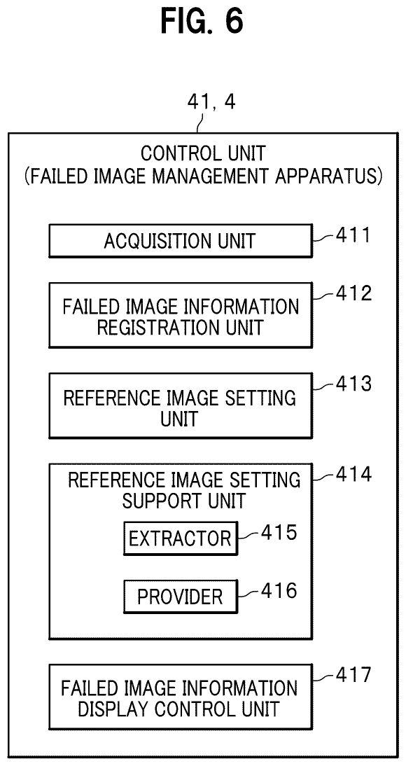

FIG. 6 is a block diagram showing functions relating to the failed image management achieved by the failed image management apparatus 4.

The control unit 41 of the failed image management apparatus 4 includes, for example, an acquisition unit 411, a failed image information registration unit 412, a reference image setting unit 413, a reference image setting support unit 414, and a failed image information display control unit 417.

The reference image setting support unit 414 includes an extractor 415 and a provider 416.

The acquisition unit 411 acquires a medical image (including supplementary information), imaging result record information, and the like from an external apparatus (the inspection apparatus 2, the console 3, or the like) via the communication unit 44 (see FIG. 3).

The failed image information registration unit 412 registers a medical image and information on a failed image (failed image information).

Registering a medical image and failed image information by the failed image information registration unit 412 is, for example, as follows.

### Registration of Failed Image Information and the Like

When an imaged region of a patient is imaged by the inspection apparatus 2 in accordance with an imaging direction, an imaging technician checks whether a medical image is a diagnostic image or a failed image through the console 3.

The console 3 transmits an imaging result record information (see FIG. 2) generated for each inspection to the imaging failure management apparatus 4 together with an image file of the medical image captured by the inspection apparatus 2.

The acquisition unit 411 acquires, through the console 3 via the communication unit 44, image files of one or a plurality of medical images generated in the same inspection (according to the same imaging order) of the same patient and imaging result record information corresponding to the inspection.

The failed image information registration unit 412 stores the image file of each medical image in the same inspection of the same patient acquired through the console 3 into a file storage area 453 of the storage unit 45, and stores information on each medical image into the image management table 451 (see FIG. 4) of the storage unit 45.

Specifically, the failed image information registration unit 412 stores, in the image management table 451, information acquired from incidental information of the medical image as a new record in association with the medical image.

The information to be registered by the failed image information registration unit 412 includes, for example, an image UID, a patient ID, a name, sex, a date of birth, an inspection UID, an imaged region, an imaging direction, an imaging date and time, and an imaging technician.

In addition, the failed image information registration unit 412 also stores a storage location of the image file of the medical image in the “file path” field of the record corresponding to the medical image in the image management table 451.

Furthermore, the failed image information registration unit 412 acquires, from the imaging result record information, the inspection UID, the image UID of the diagnostic image, the image UID of the k-th failed image, and the imaging failure reason of the k-th failed image (k=1, 2, . . . , n).

The failed image information registration unit 412 stores “0” in the “failed image classification” field of the record corresponding to a medical image whose value of an “image UID” field in the image management table 451 is the “image UID of diagnostic image”.

In yet addition, the failed image information registration unit 412 stores “k” in the “failed image classification” field and stores the “imaging failure reason of the k-th failed image” in the “failed image reason” field in a record corresponding to a medical image whose value of the “image UID” field of the image management table 451 is the “image UID of the k-th failed image”.

The reference image setting unit 413 illustrated in FIG. 6 sets a reference image on the basis of information input by a person (e.g., an imaging technician or a doctor in each medical facility).

The setting of the reference image by the reference image setting unit 413 is, for example, as follows.

### Reference Image Setting

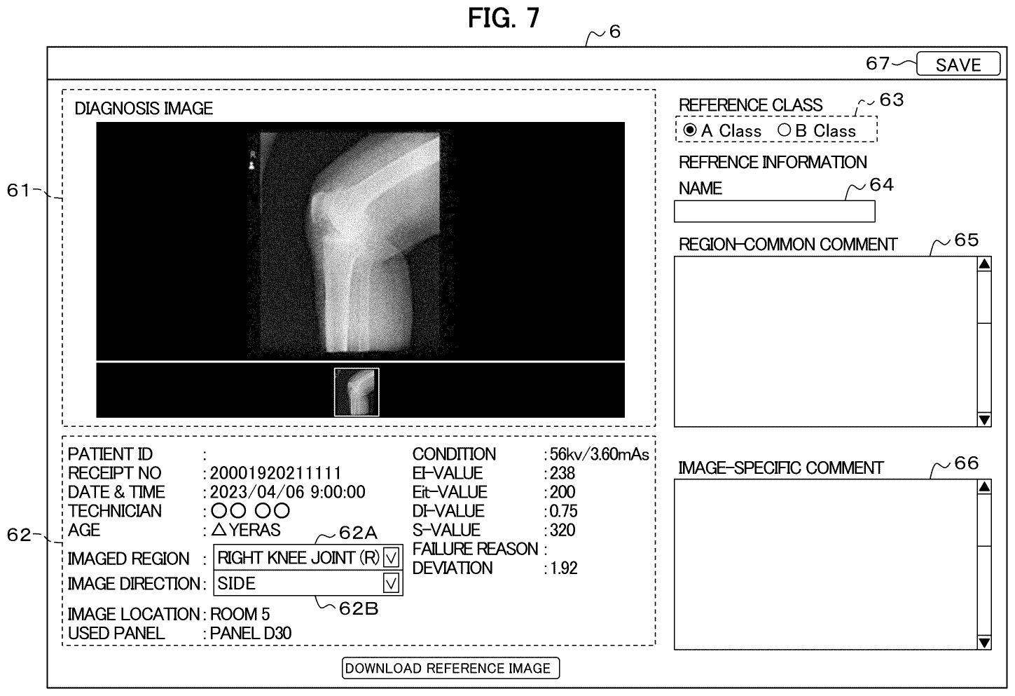

FIG. 7 is a diagram illustrating an example of a reference image setting screen 6 displayed on the display unit 42 of the rejection management apparatus 4.

The reference image setting screen 6 is a screen used for setting a reference image.

The reference image setting screen 6 includes an image area 61, a detailed information area 62, a reference image class selection area 63, a name input area 64, a region common comment input area 65, an image specific comment input area 66, a save button 67, and the like.

The image area 61 displays a diagnostic image specified by the user as a reference image.

The detailed information area 62 displays detailed information on the diagnostic image specified as the reference image.

The detailed information area 62 includes an imaged region selection area 62A and an imaging direction selection area 62B.

The imaged region selection area 62A is an area for selecting an imaged region of the reference image.

The imaging direction selection area 62B is an area for selecting an imaging direction of the reference image.

By selecting an imaged region and an imaging direction in the imaged region selection area 62A and the imaging direction selection area 62B, a reference image can be specified for each imaged region and imaging direction.

The reference image class selection area 63 is an area for selecting “A class” or “B class” for the reference image.

The name input area 64 is an area for inputting the name of the reference image.

The region-common comment input area 65 is an area for inputting a comment common to the imaged regions of the reference image.

The image-specific comment input area 66 is an area for inputting a comment specific to the reference image.

The save button 67 is a button for giving instructions to save the reference image set on the reference image setting screen 6.

For example, in a state where no image is displayed in the image area 61, the user clicks the image area 61 by an operation through the operation unit 43.

In response to the above operation, the reference image setting unit 413 causes the display unit 42 to display a list of medical images based on the image management table 451 in the storage unit 45.

Here, the reference image setting unit 413 may cause the display unit 42 to display only the diagnostic image (failed image classification: 0).

When the user designates a diagnostic image to be set as a reference image from a list through an operation from the operation unit 43, the reference image setting unit 413 acquires the “file path” of the designated medical image (reference image) from the image management table 451.

Next, the reference image setting unit 413 acquires the image file of the reference image from the file storage area 453 based on the “file path” and causes the reference image to be displayed in the image area 61.

The control unit 41 also displays, in the detailed information area 62, information included in the supplementary information on the reference image and information on the reference image managed in the image management table 451.

A user selects an imaged region in the imaged region selection area 62A and an imaging direction in the imaging direction selection area 62B through an operation from the operation unit 43.

In addition, the user selects “A class” or “B class” in the reference image class selection area 63 through an operation from the operation unit 43.

Furthermore, the user inputs the name of the reference image, the region-common comment, and the image-specific comment, respectively in the name input area 64, the region-common comment input area 65, and the image-specific comment input area 66 by operating the operation unit 43.

When the user presses the save button 67 through an operation on the operation unit 43, the reference image setting unit 413 saves the set content in a reference image setting table 452 (see FIG. 5) in the storage unit 45. Specifically, the reference image setting unit 413 stores the imaging region, the imaging direction, the image UID of the reference image, the reference image type, the name, the site-common comment, and the image-specific comment with association between them in the reference image setting table 452.

The reference image setting support unit 414 illustrated in FIG. 6 supports setting of a reference image. The support herein includes automatically setting a reference image without depending on a setting operation of the reference image by a human and helping the setting operation of the reference image by a human (providing a candidate for the reference image, and the like).

The reference image setting support unit 414 includes an extractor 415 and a provider 416.

The extractor 415 extracts an appropriate reference image (or a candidate for the reference image) from a medical image captured by the inspection apparatus 2.

Specifically, the extractor 415 extracts the appropriate reference image (or the candidate for the reference image) from diagnostic images to be used for diagnosis.

The provider 416 provides the user with a result of the extraction by the extractor 415.

As a method of providing the extraction result, for example, the extracted reference image (or the candidate of the reference image) itself may be provided to the user, or information (for example, an image UID) regarding the extracted reference image (or the candidate of the reference image) may be provided.

Conditions and timings under which the reference image setting support unit 414 automatically sets a reference image (or provides candidates for a reference image) are not particularly limited.

For example, the reference image setting support unit 414 may automatically set a reference image (or provide a candidate for a reference image) when the user sets no reference image.

The extractor 415 may provide the user with a candidate image for the reference image instead of the reference image.

The number of candidate images to be provided to the user may be plural.

In the case of providing a plurality of candidate images to the user, for example, the user checks the plurality of candidate images and selects one candidate image determined to be optimum for the reference image.

The provider 416 sets the candidate image selected by the user as the reference image.

For example, the extractor 415 extracts a reference image (or a candidate image) from a diagnostic image on the basis of evaluation information obtained when evaluating whether the image can be used for diagnosis.

The evaluation of whether an image can be used for diagnosis may be, for example, a numeric value representing the appropriateness of the diagnostic image (degree of appropriateness).

The reference image setting support unit 414 automatically assigns, to each diagnostic image, a numerical value indicating the appropriateness of the diagnostic image and uses the numerical value when extracting a reference image (or a candidate image).

An example of the evaluation information is an evaluation of a diagnostic image by a doctor who has performed interpretation of the image.

For example, the acquiring unit 411 acquires information on a doctor's evaluation of a diagnostic image from a medical image management system (PACS) or a reporting system, and the extractor 415 extracts a reference image (or a candidate image) on the basis of the acquired information on the evaluation.

Another example of the evaluation information is an evaluation as to whether or not it can be used in an academic conference or the like.

For example, information selected by a doctor as an image desired to be used in an academic conference or the like is obtained in a conference, and the extractor 415 acquires a reference image (or a candidate image) based on the selection of the doctor.

The reference image setting support unit 414 may obtain the evaluation information from at least one of information on an imaging region of a patient, information on an imaging direction, an imaging order information, patient information, and information on the diagnostic image, or a combination thereof.

The extractor 415 extracts the reference image or the candidate image from the diagnostic image on the basis of the obtained evaluation information.

The imaging order information includes information on an imaging range of a patient.

Further, the patient information includes information such as the age, sex, and body type of the patient.

In addition, the information of the diagnostic image includes information on the imaging range and the deviation of the posture of the patient (deviation information of the posture of the patient).

An example of the deviation information is a deviation amount between a medial condyle and a lateral condyle of a knee joint.

Image diagnosis of a side surface of a knee joint requires an image captured from an angle at which a joint cavity is widely viewed, but the knee joint is also a portion in which a positional deviation tends to occur in positioning at a time of imaging.

The acquisition unit 411 receives the deviation information (for example, a deviation amount) of the patient's body posture from an external apparatus such as the inspection apparatus 2 and extracts a reference image (or a candidate image) based on the deviation information received by the extractor 415.

The deviation amount can be obtained using, for example, a known image analysis technique.

A method of calculating the deviation amount is not particularly limited, and it is also possible to calculate the deviation amount using an artificial intelligence (AI) technology.

Note that the acquisition unit 411 may receive the deviation amount of the joint in the image from an external apparatus such as the inspection apparatus 2.

In that case, the extractor 415 may score an appropriateness of the diagnostic image using the deviation amount and extract a reference image (or a candidate image) based on the score.

Alternatively, the reference image setting support unit 414 may obtain the displacement amount from the diagnostic image by itself.

The failed image information display control unit 417 illustrated in FIG. 6 causes information on a medical image to be displayed to a user.

For example, the failed image information display control unit 417 causes the reference image to be displayed on the same screen as that at the time of imaging of the console 3.

Furthermore, the failed image information display control unit 417 causes a reference image corresponding to the same imaging method as that for the failed image to be displayed together with the failed image and the diagnostic image.

The failed image information display control unit 417 preferably causes the display unit 42 to display the failed image, the diagnostic image, and the reference image in a comparable manner.

The term “comparable” only requires a state in which the user can compare the three images, i.e., the failed image, the diagnostic image, and the reference image, and includes displaying the images in different windows.

The failed image information display control unit 417 causes, for example, the failed image, the diagnostic image, and the reference image to be displayed side by side in one window.

The display processing of the failed image information by the failed image information display control unit 417 is, for example, as follows.

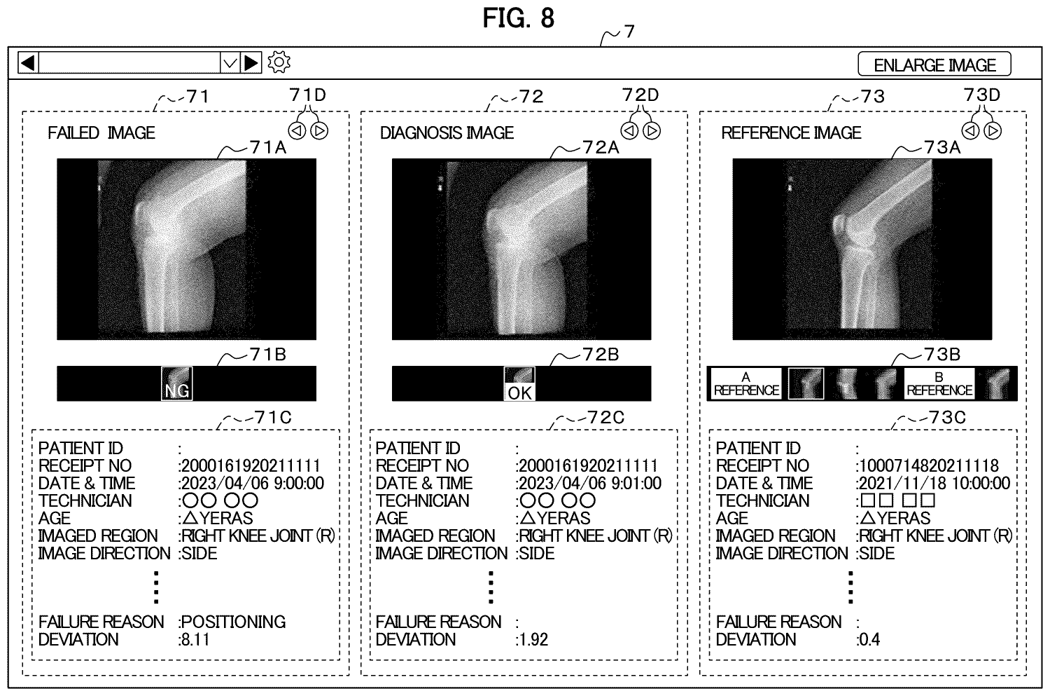

FIG. 8 is a diagram illustrating an example of a comparison display screen 7 displayed on the display unit 42.

The comparison display screen 7 is a screen that is used in a case in which a doctor gives an imaging technician lectures such as how to capture an image in an education conference or the like.

The comparison display screen 7 includes a failed image information display area 71, a diagnostic image information display area 72, a reference image information display area 73, and the like.

The failed image information display area 71 includes an image area 71A, a thumbnail area 71B, a detailed information area 71C, and position adjustment buttons 71D.

In the image area 71A, a failed image is displayed.

In the thumbnail region 71B, a thumbnail image of a failed image is displayed.

If there are a plurality of failed images for the same inspection of the same patient, thumbnail images corresponding to the plurality of failed images are displayed in the thumbnail area 71B, and a failed image corresponding to the thumbnail image selected in the thumbnail area 71B is displayed in the image area 71A.

The detailed information area 71C displays detailed information (information included in the incidental information of the failed image, information on the failed image managed in the image management table 451, and the like) on the failed image displayed in the image area 71A.

It is assumed that the diagnostic image information display area 72 or the reference image information display area 73 exists on the left side of the failed image information display area 71.

The position adjustment button 71D on the left (leftward button) is a button for switching a display position of the failed image information display area 71 with that of the diagnostic image information display area 72 or the reference image information display area 73 adjacent on the left.

Alternatively, it is assumed that the diagnostic image information display area 72 or the reference image information display area 73 exists on the right side of the failed image information display area 71.

The position adjustment button 71D on the right side (rightward button) is a button for interchanging the display position of the failed image information display area 71 with that of the diagnostic image information display area 72 or the reference image information display area 73 adjacent on the right.

The diagnostic image information display area 72 has the same configuration as that of the failed image information display area 71.

The diagnostic image information display area 72 includes an image area 72A, a thumbnail area 72B, a detailed information area 72C, and a position adjustment buttons 72D.

In the image area 72A, a diagnostic image is displayed.

In the thumbnail region 72B, thumbnail images of diagnostic images are displayed.

In the detailed information area 72C, there is displayed detailed information (information included in supplementary information on the diagnostic image, information on the diagnostic mage managed in the image management table 451, and the like) of the diagnostic image displayed in the image area 72A.

It is assumed that the failed image information display area 71 or the reference image information display area 73 exists on the left of the diagnostic image information display area 72.

The position adjustment button 72D on the left side (leftward button) is a button for interchanging the display position of the diagnostic image information display area 72 with that of the failed image information display area 71 or the reference image information display area 73 adjacent on the left.

It is assumed that the failed image information display area 71 or the reference image information display area 73 exists on the right side of the diagnostic image information display area 72.

The position adjustment button 72D on the right side (rightward button) is a button for interchanging the display position of the diagnostic image information display area 72 with that of the failed image information display area 71 or the reference image information display area 73 adjacent on the right.

The reference image information display area 73 has the same configuration as that of the failed image information display area 71.

The reference image information display area 73 includes an image area 73A, a thumbnail area 73B, a detailed information area 73C, and a position adjustment buttons 73D.

A reference image is displayed in the image area 73A.

In the thumbnail area 73B, the thumbnail images of the reference images are displayed with the A reference and the B reference distinguished from each other.

When there are a plurality of reference images, thumbnail images corresponding to the plurality of reference images are displayed in the thumbnail area 73B, and a reference image corresponding to the thumbnail image selected in the thumbnail area 73B is displayed in the image area 73A.

The detailed information area 73C displays detailed information (information included in the supplementary information on the reference image, information on the reference image managed in the image management table 451, and the like) on the reference image displayed in the image area 73A.

It is assumed that the failed image information display area 71 or the diagnostic image information display area 72 exists on the left of the reference image information display area 73.

The position adjustment button 73D (leftward button) on the left side is a button for interchanging the display position of the reference image information display area 73 with that of the left adjacent failed image information display region 71 or diagnostic image information display region 72.

It is assumed that the failed image information display area 71 or the diagnostic image information display area 72 exists on the right side of the reference image information display area 73.

The position adjustment button 73D on the right side (rightward button) is a button for interchanging the display position of the reference image information display area 73 with that of the failed image information display region 71 or the diagnostic image information display region 72 on the right side.

On the comparison display screen 7, the failed image, the diagnostic image, and the reference image are displayed side by side in one window.

The arrangement of the failed image, the diagnostic image, and the reference image to the left, center, and right positions can be changed by the position adjustment buttons 71D, 72D, and 73D.

For example, arranging the failed image and the reference image adjacent to each other facilitates recognition of how much the failed image is different from the reference image.

### Operation of Imaging Failure Management System of Embodiment

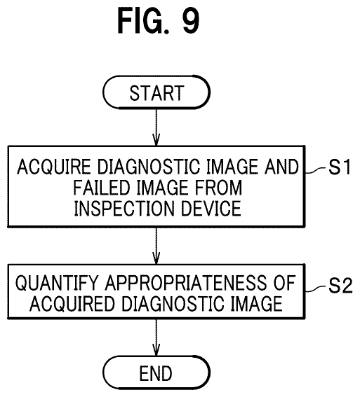

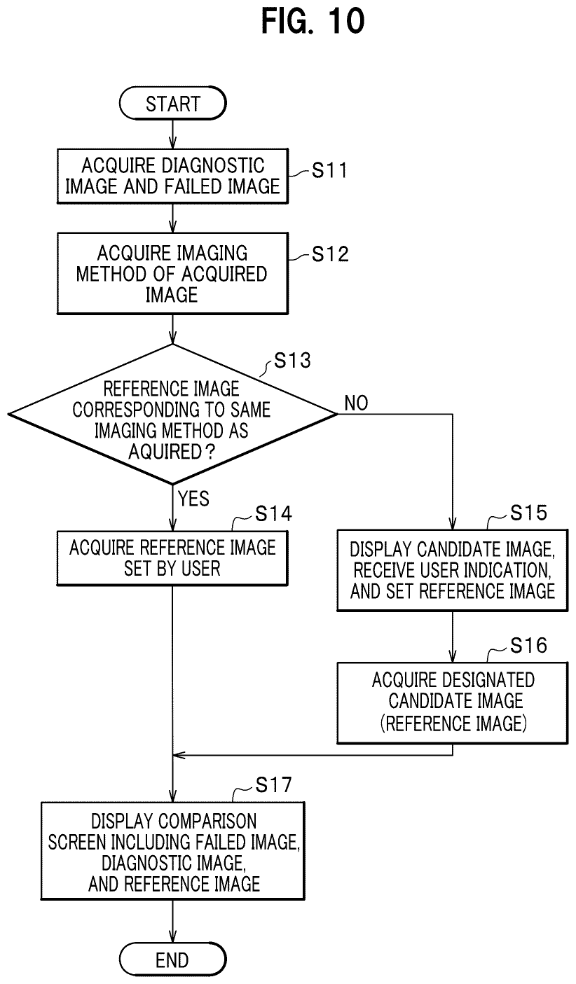

FIGS. 9 and 10 are diagrams illustrating an example of processing related to support for setting a reference image in the failed image management system 1.

FIG. 9 illustrates processing performed when the failed image management apparatus 4 acquires a diagnostic image, and FIG. 10 illustrates processing performed when the failed image management apparatus 4 is instructed to display the comparison display screen 7.

### Operation When Acquiring Diagnostic Image

As illustrated in FIG. 9, when imaging is performed, the acquisition unit 411 of the failed image management apparatus 4 acquires a diagnostic image and a failed image from the inspection apparatus 2 (step S1).

The failed image is acquired when there is a case of failing to capture an image.

Subsequently, the reference image setting support unit 414 quantifies an appropriateness of the acquired diagnostic image (step S2).

For example, the reference image setting support unit 414 quantifies the appropriateness of the diagnostic image based on the deviation amount.

Alternatively, the reference image setting support unit 414 may acquire information on a doctor's evaluation of the diagnostic image from a medical image management system (Picture Archiving and Communication System (PACS)) or a reporting system, and quantifies the appropriateness of the diagnostic image on the basis of the acquired evaluation.

Note that the reference image setting support unit 414 may have selected a diagnostic image having a high appropriateness as a reference image or a candidate image of the reference image in advance.

### Operation When Instructed to Display Comparison Display Screen

As illustrated in FIG. 10, upon receiving an instruction to display the comparison display screen 7, the failed image information display control unit 417 of the failed image management apparatus 4 acquires a diagnostic image and a failed image to be displayed (step S11).

More specifically, the failed image information display control unit 417 acquires the “file path” of the target image from the image management table 451 and acquires the image file from the file storage area 453 of the storage unit 45 based on the “file path”.

The failed image is acquired when there is a case of failing to capture an image.

Subsequently, the failed image information display control unit 417 acquires an imaging method of the acquired images (diagnostic image and failed image) (step S12).

The imaging method includes an imaged region and an imaging direction.

To be specific, the failed image information display control unit 417 acquires the “imaged region” and the “imaging direction” included in a record corresponding to the image acquired in step S11 in the image management table 451.

Subsequently, the failed image information display control unit 417 determines whether or not there is a reference image corresponding to the same imaging method as that of the image to be displayed (step S13). Specifically, the failed image information display control unit 417 refers to the reference image setting table 452 in the storage unit 45 and determines whether or not there is a record corresponding to a combination of the same “imaged region” and the same “imaging direction” as that of the image to be processed.

If there is a reference image corresponding to the same imaging method in step S13 (“YES” in step S13), the failed image information display control unit 417 acquires a reference image that has been already registered (step S14).

The already-registered reference image is, for example, a reference image preset by a user.

Note that if there are a plurality of reference images corresponding to the same imaging method as that of the image to be processed, each of the plurality of reference images is acquired.

If no reference image corresponding to the same imaging method is found in step S13 (“NO” in step S13), the reference image setting support unit 414 displays candidates for the reference image (candidate images) on the display unit 42 and receives an indication from the user (step 15).

The reference image setting support unit 414 extracts a plurality of candidate images from the diagnostic images, for example, based on the quantified appropriateness of the diagnostic images and displays the candidate images extracted.

The user designates one candidate image as a reference image from among the plurality of displayed candidate images by visual determination.

Then, the reference image setting support unit 414 sets the candidate image specified by the user as the reference image for the image capturing method (step S15).

One optimal candidate image may be set as the reference image without displaying the candidate images to receive the designation by the user.

In this case, the processing of step S15 is unnecessary.

Subsequent to step S15, the failed image information display control unit 417 acquires the candidate image (reference image) designated and set in step S15 (step S16).

Then, the failed image information display control unit 417 creates the comparison display screen 7 using the reference image acquired in step S14 or step S16, and causes the display unit 42 to display the created comparison display screen 7 (step S17).

The comparison display screen 7 displays, for example, the failed image, the diagnostic image, and the reference image side by side in one window.

The failed image management apparatus 4 according to the embodiment of the present invention configured as described above has the following effects.

That is, the failed image management apparatus 4 according to the embodiment includes the extractor 415 and the provider 416.

The extractor 415 extracts an appropriate reference image (or a candidate image for a reference image) from diagnostic images used for diagnosis.

The provider 416 provides a user with a result of extraction by the extractor 415.

Therefore, it is possible to reduce the user's labor required for registration of the reference image.

The extractor 415 extracts, for example, a reference image (or a candidate image for the reference image) based on evaluation information.

An example of the evaluation information is an evaluation on a diagnostic image by a doctor who has performed interpretation of the diagnostic image.

In addition, an example of the evaluation information is an evaluation as to whether or not it can be used in an academic conference or the like.

In this case, an evaluation of a doctor who is a final checker of the image can be reflected to a criterion for the evaluation.

Furthermore, the extractor 415 uses a quantitative deviation amount for the extraction of the reference image (or the candidate image for the reference image), which allows standardizing the criterion for selecting the reference image (or the candidate image for the reference image).

Although the embodiments of the present invention have been described and illustrated in detail, the disclosed embodiments are for the purpose of illustration only and not for limitation. The scope of the present invention should be interpreted using terms of the appended claims.

### DESCRIPTION OF REFERENCE SIGNS

1: Failed image management system (Reference image providing system)
2: Inspection apparatus
3: Console
4: Failed image management apparatus (reference image providing apparatus)
41: Control unit
411: Acquiring unit
412: Failed image information registration unit
413: Reference image setting unit
414: Reference image setting support unit
415: Extractor
416: Provider
417: Failed image information display control unit
42: Display unit
43: Operation unit
44: Communication unit
45: Storage unit
451: Image management table
452: Reference image setting table
453: File storage area
6: Reference image setting screen
61: Image area
62: Detailed information area
63: Reference image class selection area
64: Name input area
65: Region-common comment input area
66: Image-specific comment input area
67: Save button
7: Comparison display screen
71: Failed image information display area
72: Diagnostic image information display area
73: Reference image information display area

What is claimed is:

1. A reference image providing apparatus comprising a processor:
the processor configured to execute functions of:
an extractor that extracts a reference image or a candidate image of the reference image from diagnostic images used for diagnosis, the reference image serving as an imaging reference for determining whether or not an image can be used for diagnosis; and
a provider that provides an extraction result.

2. The reference image providing apparatus according to claim 1, wherein
the extractor extracts the reference image or the candidate image when no reference image is set.

3. The reference image providing apparatus according to claim 1, wherein
when receiving an instruction for the candidate image that the provider provides, the provider sets the candidate image as the reference image.

4. The reference image providing apparatus according to claim 1, wherein
the extractor acquires evaluation information on whether or not an image can be used for diagnosis to extract the reference image or the candidate image from the diagnostic image on the basis of the evaluation information.

5. The reference image providing apparatus according to claim 4,
wherein the evaluation information is information based on an evaluation result of the diagnostic image performed by a doctor.

6. The reference image providing apparatus according to claim 1, wherein
the extractor:
obtains evaluation information resulted by evaluating availability of the image for diagnosis from at least one of information on an imaged region of a patient, information on an imaging direction, imaging order information, patient information, and information on the diagnostic image; and
extracts the reference image or the candidate image from the diagnostic images on a basis of the obtained evaluation information.

7. The reference image providing apparatus according to claim 6, wherein
the information on the diagnostic image is deviation information of body posture of the patient.

8. A reference image providing method implemented by a processor, comprising:
extracting a reference image or a candidate image for the reference image from diagnostic images, the reference image serving as an imaging reference for determining whether or not an image can be used for diagnosis;
and providing a result of the extracting.

9. A non-transitory computer-readable storage medium storing a reference image providing program that, when executed by a computer, causes the computer to function as:
an extractor that extracts a reference image or a candidate image for the reference image from diagnostic images, the reference image serving as an imaging reference for determining whether or not an image can be used for diagnosis; and
a provider that provides a result that the extractor extracts.

10. A reference image providing system comprising:
the reference image providing apparatus according to claim 1, and
an inspection apparatus including a processor configured to execute a function of capturing the diagnostic image.
